(19) United States
(12) Patent Application Publication
(10) Pub. No.: US 2012/0192867 A1
(43) Pub. Date: Aug. 2, 2012

(54) PATIENT-CONTROLLED VENTILATION

(75) Inventors: STEPHEN LEWIS, Atlanta, GA (US); Cheryl Lewis, Atlanta, GA (US); Thomas Westfall, Riverside, CA (US); Terry Blansfield, Orange, CA (US)

(73) Assignee: CareFusion 303, Inc., San Diego, CA (US)

(21) Appl. No.: 13/018,248

(22) Filed: Jan. 31, 2011

(51) Int. Cl. A61M 16/00

(52) U.S. Cl. 128/204.21

(57) ABSTRACT

A method of controlling a ventilator is disclosed that includes the steps of providing a patient with a ventilator patient control interface through which a patient controls at least one control parameter of a ventilator and configuring a processor to control the ventilator in response to the ventilator patient control interface such that the patient controls the at least one control parameter of the ventilator in accordance with pre-set limits on changes to the at least one control parameter.

### BACKGROUND

1. Field

The present disclosure generally relates to systems and methods providing mechanical ventilation to assist a patient, and, in particular, relates to control of the ventilator settings by the patient.

2. Description of the Related Art

People who have been seriously injured or undergone major surgery may have difficulty in breathing on their own. In order to ensure that sufficient oxygen is available in the lungs for absorption, a ventilator may be used to mechanically assist or replace spontaneous breathing. Positive-pressure ventilators work by increasing the patient's airway pressure through a patient device such as a mask or an endotracheal or tracheostomy tube. The positive pressure forces air to flow into the lungs. When the ventilator reduces the pressure, the elastic contraction of the chest wall collapses the lungs and pushes a volume of air out. The volume of air that is introduced into the lungs on each cycle is the “tidal volume.”

Patients suffering from a severe lung injury or an illness such as chronic obstructive pulmonary disease may require long-term use of a ventilator. Some patients find that certain modes of operation or settings within the mode are more comfortable than others. It is frequently possible for the caregiver to adjust the ventilator to make the patient more comfortable while maintaining the prescribed treatment protocol, although this may be a lengthy process and the settings that are most comfortable may change repeatedly during the treatment. Typically, the patient must request the caregiver to adjust the settings of the ventilator, yet the patient is not likely to know what to ask the caregiver to adjust nor how much to change the setting.

It is normally desirable to end the use of a mechanical ventilator as early as possible. Many of the current protocols for transitioning a patient off of a mechanical ventilator, or “weaning” the patient, include one or more “spontaneous breathing trials” or “weaning trials” where the ventilator support is reduced or stopped for a period of time and the patient is monitored during the trial to identify signs of distress or difficulty. If the patient is able to complete the prescribed weaning trials, the ventilator is typically removed. The response of every patient is different, however, and one patient may be ready to discontinue use of the ventilator very quickly while another patient may require multiple repetitions of the weaning trials before they are strong enough to discontinue use of the ventilator. There is no current way for a patient to influence the course of the weaning trial to complete the trial faster or slower.

### SUMMARY

The disclosed system and method describe a ventilator system that is configured to allow the patient to control at least one of the control parameters of the ventilator. In certain embodiments, the physician may prescribe a weaning protocol that comprises a series of stages leading from an initial stage associated with greater support of the patient (such as full support) by the ventilator to a final stage that is associated with readiness of the patient to discontinue use of the ventilator. Each stage comprises a set of specified values of one or more control parameters. The patient can change the ventilator from one stage to an adjacent stage in the series. Each stage may include a lock-out time period where the patient cannot change the stage in the direction towards the final stage until the lock-out time period has elapsed while operating at the current stage. The ventilator may display one or more health parameters to assure the patient that they are safe, indicators of which stage is currently in use, or progress parameters indicating the progress of the patient towards readiness to discontinue use of the ventilator to encourage the patient in moving toward the final stage. In other embodiments, the physician may specify an operating range for one or more control parameters and the patient may vary these control parameters within the operating range to maximize their comfort.

In certain embodiments, a method of controlling a ventilator is disclosed. The method comprises the steps of providing a patient with a ventilator patient control interface through which a patient controls at least one control parameter of a ventilator and configuring a processor to control the ventilator in response to the ventilator patient control interface such that the patient controls the at least one control parameter of the ventilator in accordance with pre-set limits on changes to the at least one control parameter.

In certain embodiments, a ventilation system for use by a patient is disclosed. The ventilator system comprises a patient device attached to the patient, the patient device configured to introduce gas into the lungs of the patient; a gas control module fluidically coupled to the patient device, the gas control module configured to controllably provide a gas to the patient device according to at least one operating parameter; a memory configured to store one or more executable instructions and data; a patient control interface configured to control the at least one operating parameter of the gas control module and to be accessible by the patient; and a processor coupled to the gas control module, the patient control interface, and the memory, the processor configured to retrieve the instructions and data from the memory and operate the gas control module in accordance with the retrieved instructions and data and in response to the patient control interface.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient using a positive pressure mechanical ventilator that can be used for the system of the present disclosure.

FIG. 2 illustrates an example weaning protocol structure according to certain aspects of the present disclosure.

FIG. 3 depicts an example patient control interface according to certain aspects of the present disclosure.

FIGS. 4A-4B illustrate example configurations of patient-controllable operating parameters of a ventilator configured to maximize patient comfort according to certain aspects of the present disclosure.

FIG. 5 is a flow chart of an exemplary methodology of a patient controlling a ventilator according to certain aspects of the present disclosure.

FIG. 6 is a block diagram of a ventilator configured to be controlled by a patient according to certain aspects of the present disclosure.

FIG. 7 is a block diagram of a ventilator controller configured to control the operation of a conventional ventilator according to certain aspects of the present disclosure.

### DETAILED DESCRIPTION

While positive-pressure ventilators are generally acknowledged to be uncomfortable for a patient, current ventilators do not allow the patient to control any aspect of the operation of the ventilator to improve their comfort. Similarly, while it is generally agreed that it is desirable to get a patient off of the use of a ventilator as soon as possible, current ventilators do not allow the patient to control any aspect of the weaning process such that they might complete the weaning faster. The disclosed system and methods provides patients with the ability to control certain parameters of a ventilator to maximize their comfort or participate in the weaning process as well as provide feedback to the patient to encourage and assist them in the weaning process.

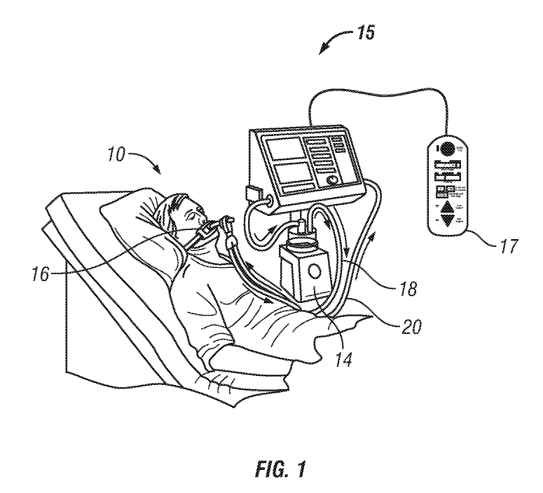

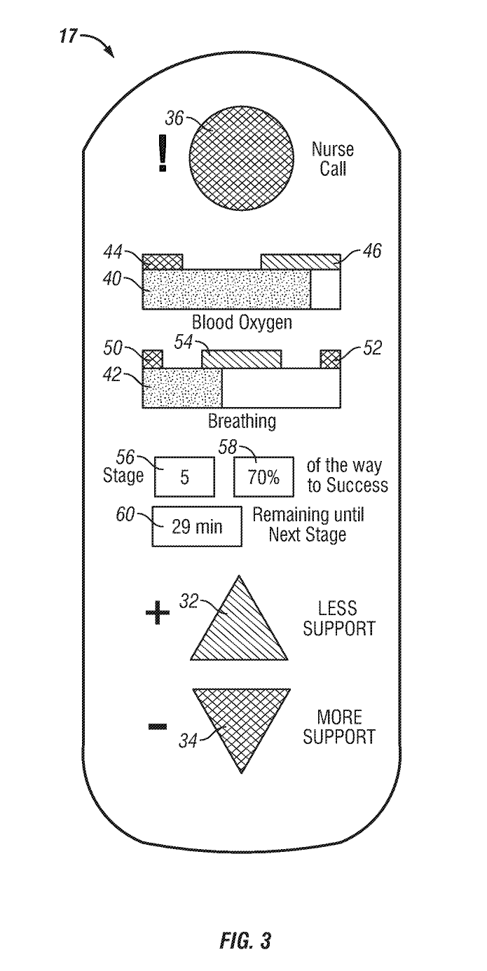

FIG. 1 depicts a patient 10 using a positive pressure mechanical ventilator 15 that can be used for the system of the present disclosure. The patient 10 is wearing a patient device 16 such as an oral endotracheal tube that is attached with straps. In other situations, alternate patient devices 16 such as a full-face or nose-and-mouth mask, a laryngeal mask, a nasal endotracheal tube, or a tracheostomy tube may be used. The ventilator 15 is, in this example, attached to the patient device 16 by a supply hose 18 and a return hose 20. Air from the ventilator 15 passes through, in this example, a humidifier 14 before entering hose 18 so that the air that is supplied to the patient 10 is humidified. The ventilator 15 also includes a patient control interface 17 that enables the patient to control certain operating parameters of the ventilator 15. The function of the patient control interface 17 is explained in more detail in FIG. 3.

Ventilators 15 may be operated in a variety of modes, including control mode ventilation, intermittent mandatory ventilation, and pressure control ventilation. Some modes, such as control mode ventilation, generate an inspiratory tidal volume while others, such as pressure control ventilation, provide a specified pressure for a specified inspiratory time. Other modes, such as pressure support ventilation or continuous positive airway pressure (CPAP), provide a constant pre-set pressure during a breath or continuously and may be used as part of the weaning process.

Ventilators have a large number of operating parameters that are used in a variety of combinations in the various modes. The settings of each parameter used in a prescribed mode may also be specified by a doctor within a wide range. Table 1 lists some example operating parameters and operational ranges.

TABLE 1

| | |
|---|---|
| Rate | 1 to 120 bpm |
| Tidal Volume | 2.0 mL to 2.5 L |
| Inspiratory Pressure | 0 to 90 cm H₂O |
| Peak Flow | 0.4 to 150 L/min |
| Inspiratory Time | 0.15 to 5.0 sec |
| % O₂ | 21 to 100% |
| PEEP | 0 to 50 cm H₂O |

where PEEP is an acronym for “positive end-expiratory pressure” and is the pressure that is maintained by the ventilator at the end of expiration to keep the airway pressure above the atmospheric pressure.

As a patient 10 recovers from the injury or surgery that led to their being placed on a ventilator 15, caregivers will often change the mode of operation of the ventilator 15 or reduce the settings to reduce the level of support provided to the patient 10 by the ventilator 15. The objective is to discontinue the use of the ventilator 15 completely as soon as possible with as little risk to the patient 10 as possible. Trials of spontaneous breathing are usually conducted and have been shown to accurately predict the success of spontaneous breathing if the patient 10 were to be removed from the ventilator 15. While the mode of ventilator operation and the settings of the operating parameters are selected by the physician depending upon the individual case, an example of a spontaneous breathing trial is to change the mode of operation to CPAP with a pressure setting of 5 cm H₂O. Such a trial, however, may be too large a change from current mode and settings of the ventilator 15. In such cases, a series of stages may be specified, wherein the nurse changes the settings to those specified in the first stage and observes the patient 10 for a specified amount of time. If the patient 10 does not exhibit signs of distress or difficulty in breathing, the nurse will change the settings to those of the next stage. If the patient 10 is able to reach the final stage without observed difficulty, the doctor may order that that patient device 16 be removed, referred to as “extubation.” This series of trial stages may take several days, especially if the patient 10 experiences difficulty or anxiety at any stage.

Ventilators 15 often also monitor patient parameters and may have alarms that can be set to trigger at certain levels. Table 2 lists examples of monitored parameters.

TABLE 2

| | |
|---|---|
| EtCO₂ | the level of carbon dioxide released at the end of expiration |
| Vti | the total volume of air inhaled, or inspired, in one minute |
| Vte | the total volume of air exhaled, or expired, in one minute |
| Spon Vt | spontaneous vidal volume |
| Ve | the total volume of air inhaled in one minute |

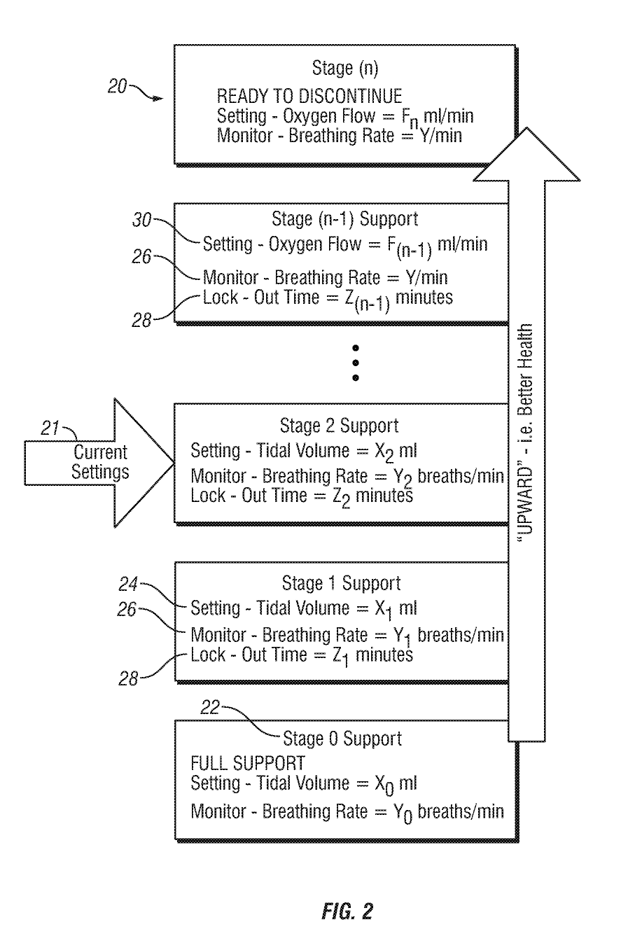

FIG. 2 illustrates an example weaning protocol structure according to certain aspects of the present disclosure. A series of stages 20 are defined, wherein each stage has a label 22 from stage 0 (zero) to stage n and the attributes of the stage listed in each box. Stage 0 is defined, in this example, as the starting point for the protocol, considered to be the level of breathing support that has been provided to the patient 10 on a continuous basis up to the start of the weaning process, which can be considered as “full support” mode of ventilator 15 operation for patient 10. Stage 0 is associated with stable and acceptable physiological parameters such as blood oxygen saturation (SpO₂). At the other end of the series is a final stage n, wherein the settings are associated with the patient being ready to discontinue use of the ventilator. If one or more intermediate stages are defined, the mode of operation and the settings of the operating parameters associated with each mode of operation may be varied from the previous stage. For the purpose of this discussion, movement between stages in a direction from initial stage 0 towards the final stage n is referred to as “upward”, as stage n is considered to be a higher level of health than stage 0, while movement between stages in the opposite direction is referred to as “downward.” In this example, the arrow 21 indicates that the ventilator is currently operating at the settings of stage 2, wherein stage 1 and stage 3 (not shown) are considered “adjacent” stages, with stage 1 considered to be a downward adjacent stage and stage 3 an upward adjacent stage. The ventilator 15 is configured, in this example, to change only stepwise from the current stage to an adjacent stage, either upward or downward.

In FIG. 2, stages 0, 1, and 2 all specify at least the setting of one operating parameter 24, tidal volume in this example, wherein this operating parameter 24 in each stage 0, 1, and 2 has a value X₀, X₁, and X₂, respectively, that may be the same as or different from the value of the adjacent stage. Each of stages 0, 1, and 2 also monitor a health parameter 26 that is, in this example, breathing rate with respective values Y₀, Y₁, and Y₂ that are alarm limits that also may be the same as or different from the value of the adjacent stage. Each of stages 1 and 2 also has, in this example, a specified lock-out time 28 having values of Z₁ and Z₂ such that the ventilator 15 must operate at that stage for at least the time period specified in the lock-out time 28 before the ventilator 15 can be changed to the next upward stage. Stage 0 does not have a lock-out time 28 as it is the baseline set of operating conditions. In some embodiments, a downward change (towards greater support) between stages is not limited by this lock-out time.

In the stages (n−1) and n that are depicted in FIG. 2, the mode of operation of the ventilator has changed and now at least one operating parameter 30 is, in this example, oxygen flow at respective flow rates F₍ₙ₋₁₎ and F₂. Stages (n−1) and n continue to monitor breaths per minute, although in certain embodiments other parameters might be monitored in addition to or instead of breathing rate. Stage (n−1) has a lock-out time 28 while stage n does not, as there is no higher stage than stage n.

In certain embodiments, the patient 10 is able to control the ventilator 15 to transition between the stages defined in the protocol of FIG. 2. The patient 10 can step upwards one stage at a time, with a minimum time between steps as defined by the respective lock-out times 28 of each stage. In some embodiments, the ventilator 15 will not step up to the next stage if a monitored parameter 26 is outside of a limit (not shown). In some embodiments, the patient 10 can step down one stage at any time. In some embodiments, the patient 10 can step down more than one stage at a time. In some embodiments, there is a second lock-out time (not shown) that specifies a minimum time between downward steps.

FIG. 3 depicts an example patient control interface 17 according to certain aspects of the present disclosure. In this example, the patient control interface 17 is a wireless handheld that is similar in size to a television remote control. This handheld 17 is configured to enable the patient 10 of FIG. 1 to participate in the weaning process wherein the ventilator 15 of FIG. 1 has been configured according to a weaning protocol such as shown in FIG. 2. There are 3 buttons on the example handheld 17—an “up” button 32 that configures the ventilator 15 to transition between stages and operate at the adjacent upward stage, a “down” button 34 that configures the ventilator 15 to transition between stages and operate at the adjacent downward stage. Button 36 is a nurse call button that replicates the function of the separate nurse-call actuator that is normally provided to patients in a hospital. These buttons may be illuminated and/or color-coded to assist the patient 10 in understanding their function or operating them at night or in reduced illumination. For instance, the up button 32 may be green, suggesting that moving up the series of weaning stages is a positive step, while the down button 34 may be yellow to suggest that it is undesirable to move down the series of weaning stages. The nurse call button 36 may be red to indicate that it is the button to push if the situation is urgent or the patient is in distress.

As using a ventilator 15 may be inherently uncomfortable and it may increase the discomfort to move upward in the weaning protocol even when the patient 10 is not at an increased risk, it may be desirable to provide assurance to the patient 10 that they are not at risk of injury. To this end, feedback is provided displaying health parameters of the patient 10 that are, in this example, the measured value of the patient's blood oxygen level 40 and the measured value of the patient's breathing rate 42. To provide an intuitive guide to the desired ranges of these health parameters, the displays 40 and 42 may have adjacent colored bars that may be red to indicate undesirable ranges and green to indicate desirable ranges of each parameter. In this example, blood oxygen 40 has a red bar 44 and a green bar 46 while breathing rate has red bars 50 and 52, as the patient's breathing rate could be undesirably high or low, as well as a green target bar 54. By examining the displays 40 and 42, the patient 10 and their family can verify that the patient is not in physical danger although they may be in discomfort.

To further encourage a patient 10 to progress through the stages of the weaning process, it may be desirable to provide feedback to the patient 10 showing how much progress that they have made toward the final stage of the weaning process. In this example, the feedback includes a display of the stage number 56 and a percentage of the progress 58 towards the final stage that is associated with the current stage. In other embodiments, display 56 may include a “X of Y” format to include the total number of stages and to show the progress. For example, the display 56 could show “5 of 7” to indicate that stage 7 is the final stage and that the patient is currently in stage 5.

In embodiments wherein one or more stages include a lock-out time during which the ventilator 15 will not change to a higher stage even if the patient 10 presses the up button 32, it may be desirable to provide feedback to the patient 10 regarding the amount of time remaining in the current lock-out period. To this end, display 60 is provided in this example to display the minutes remaining in the current lockout period. In certain embodiments, if a stage does not have a lock-out period specified, display 60 may be zero. In certain embodiments, the display 60 may change to a text term such as “READY” instead of a zero.

The patient control interface may be configured in a variety of alternate configurations without departing from the scope of this disclosure and the related claims. Alternate display devices, such as liquid crystal displays (LCDs) or color display screens, may combine multiple displays. Alternate input devices, such as a touch-screen, mouse, joystick, etc. may be used instead of the button described above. The patient control interface 17 may be provided by a device separate from the ventilator 15, such as an application running on a desktop computer or a cell phone.

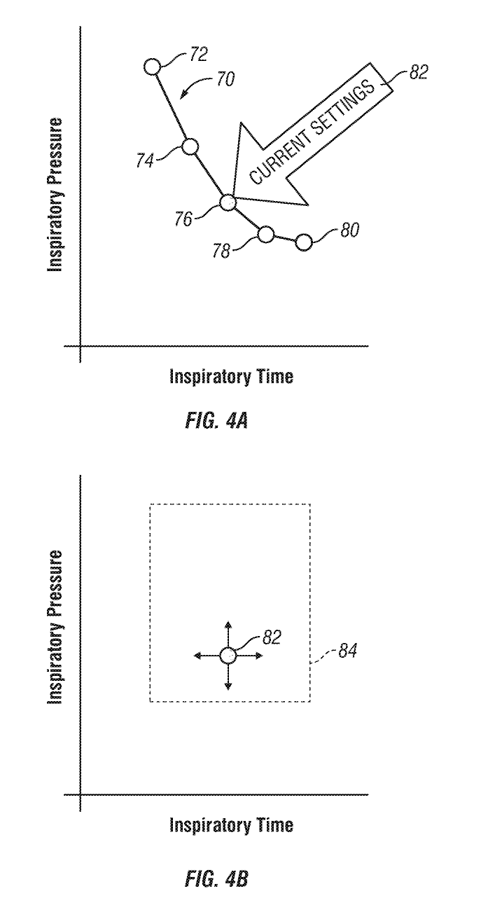

FIG. 4A-4B illustrate example configurations of patient-controllable operating parameters of a ventilator 15 configured to maximize patient comfort according to certain aspects of the present disclosure. A patient 10 of FIG. 1 may be adequately supported by a ventilator 15 of FIG. 1 operating over a range of settings of one or more operating parameters. Some of these settings, or combinations of these settings, may be more comfortable than others to a particular patient. Each patient is different, and what feels best for one patient may not be the most comfortable set of settings for another patient. While the nurses and caregivers may attempt to adjust the ventilator settings to increase the comfort of the patient, it is difficult for the patient to convey their degree of comfort to the nurse while the patient device 16 of FIG. 1 is in place. In the example of FIG. 4A, a ventilator 15 has been configured to specify combinations 72, 74, 76, 78, and 80 of inspiratory time and inspiratory pressure that are considered to be acceptable for the patient in their current condition. These combinations 72/74/76/78/80 are linked into a range 70. A patient control interface similar to that of FIG. 3 (not shown) will have up and down buttons that adjust operating parameters of the ventilator 15 from one combination, such as combination 76, to an adjacent combination, such as combination 74 or 78, within the range 70. The patient 10 may use the patient control interface to change the settings within range 70 according to their comfort without having to try and communicate with a nurse or other caregiver. In certain embodiments, range 70 may comprise only a single operating parameter while in certain other embodiments, range 70 may include a plurality of operating parameters.

In the example of FIG. 4B, the patient control interface 17 has multiple inputs that independently control the two operating parameters. The ventilator 15 has been configured by the nurse to allow the two operating parameters to continuously vary within an operating range 84. The current settings are shown as point 82 wherein the arrows indicate that the parameters may be independently varied within the range. In certain embodiments, the ventilator 15 may be configured to change one or both operating parameters in steps within the range 84.

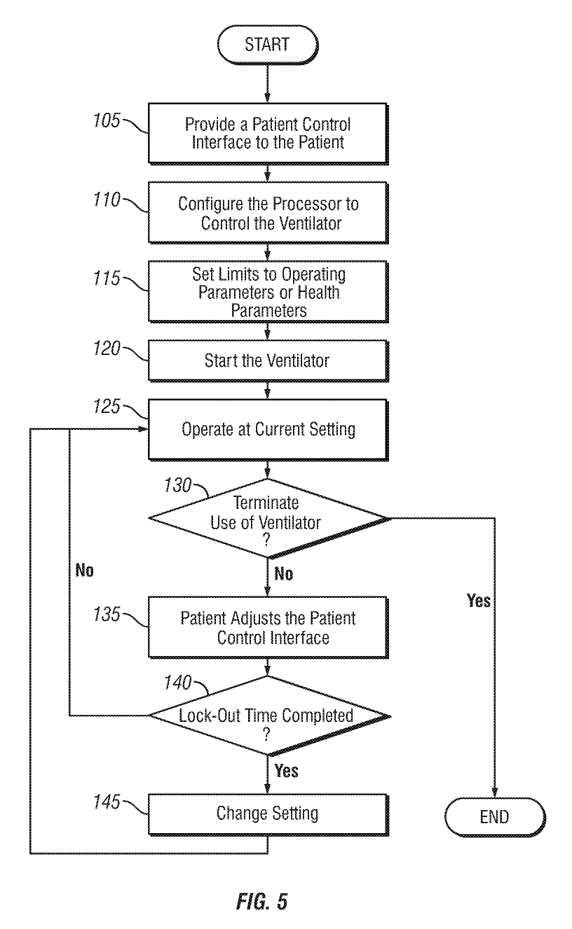

FIG. 5 is a flow chart of an exemplary methodology of a patient 10 controlling a ventilator 15 according to certain aspects of the present disclosure. The process starts in step 105 wherein a nurse, a doctor, or other caregiver provides a patient control interface, such as the handheld 17, to a patient 10 who is or will be using a ventilator 15. The nurse or other caregiver then configures the ventilator 15 in step 110 to define how the inputs of the patient control interface 17 control the ventilator 15. In certain embodiments, this may include defining one or more stages of a weaning process. In certain embodiments, this may include specifying combinations of settings that are acceptable for use by the patient 10 in their current condition. This process of defining the stages can be provided by a remote processor coupled to the ventilator 15 through a wired or wireless network. In step 115, the nurse (or local or remote processor) specifies limits to the operating parameters, such as the end combinations 72 and 80 in FIG. 3, or limits for monitored health parameters such as SpO₂ or breathing rate. This step may also include specifying what limits have alarms associated with them or what limits are associated with prevention of certain actions, such as not allowing the ventilator 15 to move to a higher stage in the weaning process if the breathing rate exceeds an upper or lower limit. Once all of the operating, safety, and other limits are configured, the nurse then starts the ventilator in step 120. In certain embodiments, the ventilator 15 may already have been operating in a non-patient-controlled mode and step 120 comprises switching the mode of operation to a patient-controlled mode. The process then moves to step 125 wherein the ventilator 15 operates at the current settings until an action is taken by either the nurse or the patient 10.

The nurse may initiate a termination of the patient-controlled operation of the ventilator 15 in step 130, whereupon the process branches along the “YES” path to “END” the patient-controlled operation of the ventilator 15. An alternate action by the nurse would be to turn off the ventilator 15, such as when the patient 10 successfully completes the weaning process and the patient device 16 is removed, which follows the same process path to “END”. If the nurse does not initiate an action, the process may then proceed to step 135 wherein the patient 10 adjusts the patient control interface 17. The process then moves to decision block 140 where, if a lock-out time has been specified and the lock-out time has not yet been completed for the current stage, the process will branch along the “NO” path back to step 125. If the lock-out time has been completed, or there is no lock-out time specified for the current mode of operation, the process moves to step 145 where the settings of the operating parameters that were specified in 110 and 115 are changed according to the patient's adjustment of the patient control interface 17 and the process then branches back to step 125 to operate at the new settings, which have become the current settings. The ventilator 15 continues to loop through the steps 125-135-140-145 until a nurse takes an action in step 130.

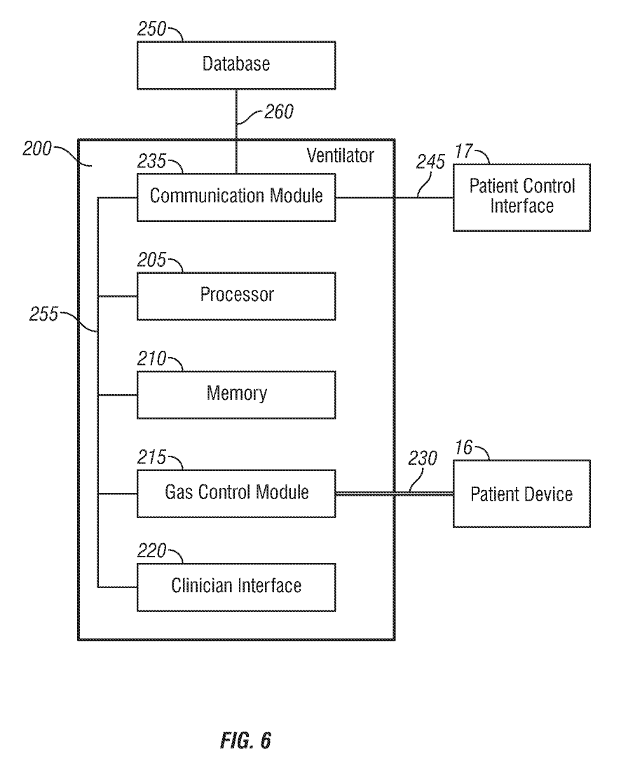

FIG. 6 is a block diagram of a ventilator 15 configured to be controlled by a patient 10 according to certain aspects of the present disclosure. The ventilator 15 is shown in this example as ventilator assembly 200, comprising a gas control unit 215, a processor 205 and memory 210, a clinician interface 220, and a communication module 235. In certain embodiments, some of these elements will be omitted while in certain other embodiments, additional elements may be incorporated into ventilator assembly 200. In certain embodiments, elements such as the clinician interface 220 may be external to the ventilator assembly 200. In certain embodiments, elements such as the clinician interface 220 may be provided by another piece of equipment such as a standard desktop computer or a handheld device such as a cellular phone. In certain embodiments, the elements shown may be combined or functions from one element may be accomplished by another element. The elements 205, 210, 215, 220, and 235 are shown as interconnected by a bus 255, enabling each element to talk to any other element on the bus. In certain embodiments, some or all of the elements 205, 210, 215, 220, and 235 may be interconnected only with one or more of the other elements by any methods of communication known to those of ordinary skill in the art, including multiple parallel buses and serial data links.

Ventilator assembly 200 is connected to a patient device 16 that may be any of the masks or intubation devices known to those of ordinary skill in the art for introducing gas into the lungs of a patient, including full-face or partial-face masks, an endotracheal tube, or a tracheotomy tube. The connection between ventilator assembly 200 and patient device 16 is, in this example, accomplished by an air hose 230 from the gas control module 215 to the patient device 16. In certain embodiments, air hose 230 includes a supply hose and a return hose (not shown separately) such that the patient's exhaled gas is returned to the ventilator assembly 200.

Ventilator assembly 200 is also coupled, in this example, from communication module 235 to a patient control interface 17 through a communication link 245. In certain embodiments, such as the wireless handheld 17 of FIG. 3, communication link 245 may be a optical or radio-frequency one-way or bidirectional link. In certain other embodiments, the patient control interface 17 may be a part of the ventilator assembly 200, an alternate screen display on the clinician interface 220, or a display on a separate computer.

In certain embodiments, the communication module 235 of ventilator 200 may be linked to an external server or database 250 through a network 250 such as an Ethernet wired or wireless network. The processor 205 may retrieve executable instructions, information on prescribed operating parameters for a specific patient 10, or other data or information related to the operation of ventilator 200 or to the patient 10. Similarly, processor 205 may transmit information to the database 250, such as a history of operation, a log of patient actions, or a record of actuations of the patient control interface 17 regardless of whether the ventilator 200 implemented the associated change.

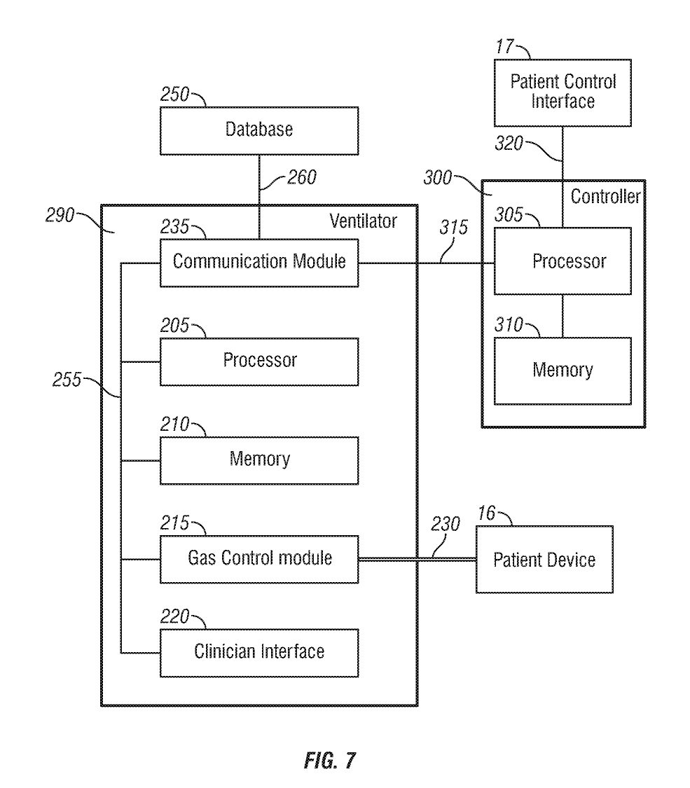

FIG. 7 is a block diagram of a ventilator controller 300 configured to control the operation of a conventional ventilator 290 according to certain aspects of the present disclosure. Ventilator 290 comprises the same elements as the ventilator 200 of FIG. 6, including the processor 205, the memory 210, gas control module 215, the clinician interface 220, and the communication module 235. The gas control module 215 is coupled through air hose 230 to the patient device 16. Processor 205 is coupled to database 250 through the communication module 235 and network 260.

In this example, a ventilator controller 300 is coupled to the conventional ventilator 290. More precisely, the processor 305 of the ventilator controller 300 is coupled through a wired or wireless communication link 315 to communication module 235 and then to the processor 205 of the conventional ventilator 290. The processor 205 of ventilator 290 is configured to allow the operating parameters of the ventilator 290 to be changed remotely by signals received by the processor 201 through communication module 235. Processor 305 is coupled to memory 310 that comprises instructions on how to adjust the operating parameters of the ventilator 290. Processor 305 is also coupled to the patient control interface 17 through a wired or wireless linkage 320, wherein the processor 305 is configured to transmit signals to the processor 205 to change the operating parameters of the conventional ventilator 290 according to the input from patient control interface 17 and the instructions stored in memory 310. In this example, controller 300 is directly attached to the conventional ventilator 290. In certain embodiments, controller 300 is remote from the conventional ventilator 290. In certain other embodiments, the communication link 315 comprises the network 260, wherein controller 300 is connected to the same network 260.

In the previous detailed description, numerous specific details have been set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

It can be seen that the disclosed embodiments of a patient-controlled ventilator provide a patient with the ability to adjust the operation of the ventilator within limits set by the doctor and other caregivers. In certain embodiments, the patient can progress at their own rate through a weaning process that includes a series of stages from full support to readiness to discontinue use of the ventilator. In certain embodiments, the patient receives feedback on their health to assure them that they are not at risk as they move through the stages of the weaning process. In certain embodiments, the patient receives positive feedback as they progress through the stages of the weaning process to encourage them to move forward as quickly as possible. In certain embodiments, there may be time lock-out periods or health parameters limits that prevent the patient from changing the ventilator to the next stage until the lock-out period has elapsed or while the health parameter is outside a limit. In certain embodiments, the patient can adjust one or more operating settings of the ventilator to improve their personal comfort.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A method of controlling a ventilator, the method comprising the steps of:
providing a patient with a patient control interface through which a patient controls at least one control parameter of a ventilator; and
configuring a processor to control the ventilator in response to the patient control interface such that the patient controls the at least one control parameter of the ventilator in accordance with pre-set limits on changes to the at least one control parameter.

2. The method of claim 1, further comprising the steps of:
measuring at least one health parameter that is associated with the current health of the patient; and
displaying the at least one health parameter.

3. The method of claim 1, wherein the step of configuring the processor further comprises specifying at least an initial value and a final value of the at least one control parameter, the initial value associated with greater support of the patient by the ventilator and the final value associated with readiness of the patient to discontinue use of the ventilator such that the processor changes the at least one control parameter between the initial value and the final value in response to the patient control interface.

4. The method of claim 3, wherein the step of configuring the processor further comprises specifying a weaning protocol comprising a series of stages, the series comprising an initial stage associated with full support of the patient by the ventilator and a final stage associated with readiness of the patient to discontinue use of the ventilator, the series having a direction of upward from the initial stage towards the final stage and downward in the opposite direction, each stage comprising a value of one or more control parameters such that the processor controls the ventilator in accordance with the values of the one or more control parameters of a current stage and stepwise changes from the current stage in the series to an adjacent stage in the series in response to the patient control interface.

5. The method of claim 4, wherein the step of configuring the processor further comprises specifying a lock-out time period for at least one stage such that the processor stepwise changes from the current stage to adjacent upward stage only after the ventilator has been operating at the current stage for the lock-out time period associated with the current stage.

6. The method of claim 5, further comprising the step of displaying the amount of time remaining in the lock-out period of the current stage.

7. The method of claim 4, wherein the step of configuring the processor further comprises specifying at least one limit for at least one monitored parameter that is associated with the health of the patient for at least one stage such that the ventilator measures the monitored parameter and stepwise changes from the current stage to the adjacent upward stage only when the at least one monitored parameter is within the at least one limit.

8. The method of claim 4, further comprising the step of displaying a stage identifier that is associated with the current stage.

9. The method of claim 4, further comprising the step of displaying an encouragement parameter that is associated with the current stage, the encouragement parameter representing a degree of progress towards the final stage.

10. The method of claim 1, wherein the step of configuring the processor further comprises selecting an operating range for the at least one control parameter such that the patient may vary the at least one control parameter within the operating range to maximize the patient's comfort.

11. The method of claim 10, wherein the step of configuring the processor further comprises selecting operating ranges for each of two or more control parameters, and further configuring the processor to define a link between the two or more control parameters and a single input of the ventilator patient control interface such that the processor adjusts the two or more control parameters according to the single input.

12. The method of claim 10, further comprising the step of displaying a setting parameter that is associated with the current value of the at least one operating parameter.

13. A ventilation system for use by a patient, comprising:
a patient device attached to the patient, the patient device configured to introduce gas into the lungs of the patient;
a gas control module fluidically coupled to the patient device, the gas control module configured to controllably provide a gas to the patient device according to at least one operating parameter;
a memory configured to store one or more executable instructions and data;
a patient control interface configured to control the at least one operating parameter of the gas control module and to be accessible by the patient; and
a processor coupled to the gas control module, the patient control interface, and the memory, the processor configured to retrieve the instructions and data from the memory and operate the gas control module in accordance with the retrieved instructions and data and in response to the patient control interface.

14. The ventilation system of claim 13, wherein:
the gas control module is further configured to measure a reported parameter that is associated with the health of the patient; and
the patient control interface is further configured to display the reported parameter.

15. The ventilation system of claim 13, wherein:
the executable instructions further comprise a weaning protocol, the weaning protocol comprising a series of stages comprising an initial stage that is associated with full support of the patient by the ventilator and a final stage that is associated with readiness of the patient to discontinue use of the ventilator, the series having a direction of upwards from the initial stage towards the final stage and downwards in the opposite direction;
each stage comprises a value of the at least one operating parameter;
a patient control interface configured to select the stage; and
the processor is further configured to operate in accordance with a current stage that is one of or between the initial stage and the final stage in response to the patient control interface.

16. The ventilation system of claim 15, wherein the processor is configured to stepwise change the current stage to an adjacent stage in the series of stages of the protocol.

17. The ventilation system of claim 16, wherein:
each stage comprises a respective minimum duration of operating time; and
the processor is further configured to stepwise change to the adjacent upward stage only after the respective minimum duration of operating time has elapsed at the current stage.

18. The ventilation system of claim 17, wherein the patient control interface is further configured to display the time remaining in the respective minimum duration of operating time at the current stage.

19. The ventilation system of claim 16, wherein the patient control interface comprises an input device that causes the processor to change from the current stage to the adjacent upward stage.

20. The ventilation system of claim 16, wherein the patient control interface comprises an input device that causes the processor to change from the current stage to the adjacent downward stage.

21. The ventilation system of claim 16, wherein:
each stage comprises a limit related to the at least one monitored parameter; and
the processor is further configured to stepwise change from the current stage to the adjacent upward stage only when the at least one monitored parameter is within the related limit.

22. The ventilation system of claim 15, wherein:
each stage comprises an identifier; and
the patient control interface is further configured to display the identifier of the current stage.

23. The ventilation system of claim 15, wherein:
each stage comprises an progress parameter that represents the degree of progress towards the final stage of the protocol; and
the patient control interface is further configured to display the progress parameter of the current stage.

24. The ventilation system of claim 13, wherein the executable instructions further comprise an operating range for the at least one control parameter such that the patient may vary the at least one control parameter within the operating range to maximize the patient's comfort.

25. The ventilation system of claim 24, wherein:
the patient control interface further comprises a single input;
the executable instructions further comprise operating ranges for each of two or more control parameters and links between the two or more control parameters and the single input; and
the processor adjusts the two or more control parameters according to the single input.

26. The ventilation system of claim 25, wherein the patient control interface is further configured to display a setting parameter that is associated with the current value of the single input.

27. A computer-readable medium having computer-executable instructions stored thereon for execution by a processor to perform a method of controlling a ventilator, the method comprising the steps of:
providing a patient with a patient control interface through which a patient controls at least one control parameter of a ventilator; and
configuring a processor to control the ventilator in response to the patient control interface such that the patient controls the at least one control parameter of the ventilator in accordance with pre-set limits on changes to the at least one control parameter.

28. The computer-readable medium of claim 27, further comprising the steps of:
measuring at least one health parameter that is associated with the current health of the patient; and
displaying the at least one health parameter.

29. The computer-readable medium of claim 27, wherein the step of configuring the processor further comprises specifying at least an initial value and a final value of the at least one control parameter, the initial value associated with greater support of the patient by the ventilator and the final value associated with readiness of the patient to discontinue use of the ventilator such that the processor changes the at least one control parameter between the initial value and the final value in response to the patient control interface.

30. The computer-readable medium of claim 29, wherein the step of configuring the processor further comprises specifying a weaning protocol comprising a series of stages, the series comprising an initial stage associated with full support of the patient by the ventilator and a final stage associated with readiness of the patient to discontinue use of the ventilator, the series having a direction of upward from the initial stage towards the final stage and downward in the opposite direction, each stage comprising a value of one or more control parameters such that the processor controls the ventilator in accordance with the values of the one or more control parameters of a current stage and stepwise changes from the current stage in the series to an adjacent stage in the series in response to the patient control interface.

31. A ventilator controller configured to control a conventional ventilator, the ventilator controller comprising:
a memory configured to store one or more executable instructions and data;
a patient control interface configured to control the at least one operating parameter of the ventilator and to be accessible by the patient; and
a processor coupled to the ventilator, the patient control interface, and the memory, the processor configured to retrieve the instructions and data from the memory and operate the ventilator in accordance with the retrieved instructions and data and in response to the patient control interface.

32. The ventilator controller of claim 31, wherein:
the executable instructions further comprise a weaning protocol, the weaning protocol comprising a series of stages comprising an initial stage that is associated with full support of the patient by the ventilator and a final stage that is associated with readiness of the patient to discontinue use of the ventilator, the series having a direction of upwards from the initial stage towards the final stage and downwards in the opposite direction;
each stage comprises a value of the at least one operating parameter;
a patient control interface configured to select the stage; and
the processor is further configured to operate in accordance with a current stage that is one of or between the initial stage and the final stage in response to the patient control interface.

33. The ventilator controller of claim 32, wherein the processor is configured to stepwise change the current stage to an adjacent stage in the series of stages of the protocol.
